(19) United States
(12) Patent Application Publication
(10) Pub. No.: US 2026/0256965 A1
(43) Pub. Date: Sep. 3, 2026

(54) THERAPEUTIC HYDROGELS

(71) Applicant: Vivos, Inc., d/b/a Advanced Medical Isotope Corporation, Kennewick, WA (US)

(72) Inventors: John Solomon GARNER, West Lafayette, IN (US); David SWANBERG, Kennewick, WA (US); Bradley WEEKS, West Richland, WA (US); James DUNCAN, Kennewick, WA (US); Michael KORENKO, Pasco, WA (US); Rebecca Anne IMMEL, Lafayette, IN (US)

(73) Assignee: Vivos, Inc., d/b/a Advanced Medical Isotope Corporation, Kennewick, WA (US)

(21) Appl. No.: 19/458,750

(22) Filed: Jan. 24, 2026

Related U.S. Application Data
(60) Provisional application No. 63/751,455, filed on Jan. 30, 2025.

(51) Int. Cl. A61K 51/12; A61K 9/00; A61K 47/34

(52) U.S. Cl. CPC A61K 51/1213; A61K 9/0024; A61K 47/34

(57) ABSTRACT

Disclosed herein are reabsorbable therapeutic hydrogels comprising PLGA-g-PEG polymers. It is believed that reabsorption of the hydrogel may allow healthy tissue to replace the hydrogel in healing tissues. It has been discovered that a molar ratio of lactic acid (LA) to glycolic acid (GA) in the PLGA-g-PEG polymer from about 65:35 to about 85:15 (LA:GA) is preferred for use in forming the reabsorbable therapeutic hydrogels described herein.

### CROSS REFERENCE TO RELATED APPLICATIONS

This application claims the benefit under 35 U.S.C. § 119 (e) of U.S. Provisional Application No. 63/751,455, filed on Jan. 30, 2025, the entire disclosure of which is incorporated herein by reference.

### TECHNICAL FIELD

The present novel technology relates generally to the preparation and use of reabsorbable therapeutic hydrogel compositions.

### BACKGROUND

One common approach to the treatment of patients with certain kinds of cancer, such as liver cancer, is to introduce radioactive particles into the patient's circulatory system, wherein the radioactive particles are targeted to the site of the cancer. Specifically, measured amounts of radioactive isotopes are injected into the patient such that they accumulate at the site of the cancer. The lodged particles thus generate a predetermined field of radiation within or proximate to the location of a cancerous tumor. The radioactive isotope is typically selected according to the type of radiation emitted and its half-life, such that the radiation has enough range to be destructive to the tumor and proximal tumor margins but does only minimal damage to adjacent healthy tissues and organs and also such that the emission of radiation lasts for only a short, predetermined duration.

One commonly used radioisotope is yttrium-90, since radioactive yttrium-90 emits 100 percent beta radiation and has a short half-life of 2.67 days. The yttrium-90 is typically incorporated in glass or resin microspheres which are suspended in a liquid medium and introduced via intra-arterial injection. However, the glass or resin formulations and mode of administration result in: a) difficulties in achieving a homogeneous distribution of particles within the tumor (and thus not treating the patient with a known and controlled radiation dosage); and b) difficulties in concentrating and sequestering all of the radioisotope at the tumor site allowing significant amounts of the particles to migrate away from the tumor site and deliver radiation to normal healthy tissues.

Various means have been employed to incorporate the radioisotopes in microspheres, such as those made of resin or crystalline ceramic cores with radioactive materials coated thereunto. However, whenever a microsphere comprises a core material having an external surface coating which contains the radioactive isotope there is a risk that the radioactive coating may separate from the underlying microsphere core. Any mechanical breakage of the coating can release unwanted radioactivity to other parts of the patient's body, which is highly undesirable. Further disadvantages are presented by the special handling and precautions that are necessary to coat a radioactive isotope onto a crystalline ceramic core, or to label or adsorb the isotope onto an ion exchange resin.

In still another application, microspheres have been prepared comprising a ceramic material and having a radioactive isotope incorporated into the ceramic material. While the inadvertent release of radioactive isotopes from a radioactive coating into other parts of the human body is reduced or eliminated by incorporating the radioisotopes into ceramic spheres, the latter product form is not without its disadvantages. Processing of these ceramic particles is dangerous because potentially volatile radioactivity must be added to ceramic melts and the microspheres must be produced and sized while radioactive. Such processing steps increase the likelihood of accidental exposure of personnel and risk radioactive contamination of facilities.

Some of these drawbacks have been overcome by incorporating stable ⁸⁹Y in oxide form into glass microspheres and subsequently exposing them to neutron radiation to activate the ⁸⁹Y to ⁹⁰Y. The microspheres are then injected into the patient's hepatic artery, where they may lodge in liver tumor capillaries. Microspheres may end up in normal liver or be transported elsewhere in the body. It is difficult to track and accurately assess where the administered microspheres deposit.

An approach to overcoming these difficulties is to incorporate the radioactive particles into a hydrogel. Use of bovine serum albumin cross-linked with glutaraldehyde (BioGlue™) as the hydrogel component has been described (see for example WO2021084515). This approach suffers from the slow rate of resorption of the hydrogel which can interfere with tissue healing at the site of the excised tumor. It may be that slow resorption of BioGlue™ results from a slow rate of hydrolysis. Incorporation of radioactive particles in a thermogelling hydrogel has been described (see U.S. Pat. No. 12,201,703). Injection of the hydrogel-radioactive particle material provides a localized dosage of radiation to the target tissue. Controlling the rate of reabsorption of the hydrogel remains an important parameter for improving the treatment of solid tumors. Thus, there remains a need for a better or optimized treatment that is useful in the treatment of cancer or tumor bearing tissue, but which will not release a radioactive coating or isotope, or migrate into other parts of the body of the patient after administration, yet will be reabsorbed (i.e., biodegrades) in a time frame that avoids interference with the healing of the tumor site. The present disclosure addresses this need.

It has been surprisingly discovered that hydrogels comprising PLGA-g-PEG polymers wherein the molar ratio of lactate monomers (LA) to glycolate monomers (GA) falls outside of the range from about 65:35 (LA:GA) to 85:15 (LA:GA) do not have rheological properties useful for forming therapeutic hydrogels for use in warm-blooded patients.

The descriptions of specific embodiments of the present disclosure are presented for purposes of illustration and description. They are not intended to be exhaustive or to limit the present disclosure to the precise forms disclosed, and obviously many modifications and variations are possible considering the above description. The exemplary embodiments are chosen and described to explain the principles of the present disclosure and its practical application for purposes of enabling others who are skilled in the art and making of the product to utilize the present disclosure and various embodiments with various modifications as are suited to the particular use are contemplated.

### DETAILED DESCRIPTION

For the purpose of promoting an understanding of the principles of the novel technology and presenting its currently understood best mode of operation, reference will now be made to the non-limiting embodiments and specific language will be used to describe the same. It will nevertheless be understood that no limitation of the scope of the novel technology is thereby intended, with such alterations and further modifications in the illustrated compositions and methods and such further applications of the principles of the novel technology as illustrated therein being contemplated as would normally occur to one skilled in the art to which the novel technology relates.

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a drug” includes a plurality of such drugs, so that a drug X includes a plurality of drugs X. It is further noted that the claims may be drafted to exclude any optional element.

The term “and/or” means any one of the items, any combination of the items, or all of the items with which this term is associated.

The term “about” can refer to a variation of ±2.5%, ±5%, ±10%, ±20%, or ±25% of the value specified. For an illustrative example, “about 50 percent” can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discussed above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” It is also understood that such values inherently contain variability necessarily resulting from the standard deviations found in their respective testing measurements.

As will be understood by one skilled in the art, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

It is to be understood that where items are grouped together in a common manner, such as in a Markush group, the disclosure encompasses not only the entire group listed as a whole, but each member of the group individually and all possible subgroups of the main group. Additionally, for all purposes, the disclosure encompasses not only the main group, but also the main group absent one or more of the group members. The disclosure therefore envisages the explicit exclusion of any one or more members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting, or compared to the growth or progression that occurs by treatment of a group of cells by a single drug composition.

As used herein, “gelation temperature” refers to the temperature at which a biodegradable copolymer undergoes thermal gelation, i.e. the temperature below which the block copolymer is soluble in water and above which the block copolymer undergoes phase transition to increase in viscosity or to form a semi-solid gel. The terms “gelation temperature,” “thermal gelation temperature,” “reverse thermal gelation temperature,” and similar terms can be used interchangeably.

A “polymer solution”, “aqueous solution” and the like, when used in reference to a biodegradable copolymer contained in such solution, refers to a water-based solution having the recited copolymer dissolved therein at a functional concentration, and maintained at a temperature below the gelation temperature of the block copolymer. It is understood that a polymer solution may refer to a solution of more than one biodegradable copolymer. “Thermal gelation” is the phenomenon whereby a solution of a block copolymer spontaneously increases in viscosity, and in many instances transforms into a semisolid gel, as the temperature of the solution is increased above the gelation temperature of the copolymer. For example, the term “gel” includes both the semisolid gel state and the high viscosity state that exists above the gelation temperature. When cooled below the gelation temperature, the gel may spontaneously reverse to reform or revert back to the lower viscosity solution. In many cases. cycling between the solution and the gel may be repeated ad infinitum because the sol/gel transition does not involve any change in the chemical composition of the polymer system. It is believed that interactions creating the gel are physical in nature and do not involve the formation or breaking of covalent bonds.

A “drug delivery system” or “drug delivery composition having thermal gelation properties” refers to a polymer solution that contains a drug or combination of drugs, where the drug(s) per se can be either dissolved or colloidal), suitable for administration to a warm-blooded animal, which forms a gelled drug depot when the temperature is raised to or above the gelation temperature of the block copolymer.

A “depot” refers to a drug delivery system following administration to a warm-blooded animal, which has formed a gel upon the temperature being raised to or above the gelation temperature.

A “gel” refers to the semi-solid phase that spontaneously occurs as the temperature of the “polymer solution” is raised to or above the gelation temperature of the block copolymer.

At temperatures below the gelation temperature the copolymer may be soluble in the water phase and the composition will be a solution. At temperatures at or above the gelation temperature the copolymer will solidify to form a gel with the water phase and the composition will be a gel or semi-solid.

The term “biodegradable” means that the block copolymer can chemically break down or degrade within the body to form nontoxic components under physiological conditions. The terms “resorbable” and “reabsorbable” generally refer to a material that is biodegradable. The term “resorption” is used interchangeably with the terms “reabsorption” and “biodegradation” and may include hydrolysis of the thermogel.

The terms “gel,” “thermal gel,” and “thermogel” are used interchangeably throughout this disclosure.

Radiomedicine thermal gels, such as those containing radioactive ⁹⁰Y, emit a therapeutic intensity and amount of short-range beta radiation that can penetrate tissue to a depth of up to about several millimeters. ⁹⁰Y is a commonly used radiotherapeutic isotope. ⁹⁰Y is a high-energy beta-emitting isotope with no primary gamma; the maximum energy of the ⁹⁰Y beta particle is 2.28 MeV with a mean energy of about 0.93 MeV. ⁹⁰Y has a half-life (t_{1/2}) of 64 hours, with 94% of its radiation delivered in approximately 11 days. Typically, the concentration of radioisotope in the thermal gel and the amount of thermal gel introduced into a patient is controlled such that they will not emit an excess amount of unwanted radiation that could damage healthy tissue surrounding the target tumor. Thus, the thermal gel composition is typically engineered so that the therapeutic radioisotopes are the only constituent isotopes which emit a significant amount of alpha, beta and/or gamma radiation, and that the radioisotopes have a sufficiently short half-life that the radiation emissions are extinguished after a relatively short period of time, typically on the order of several days to a few months. Designing the thermal gel portion of the radiomedicine thermal gel to have a resorption time that is similar to the time that the radiation emissions of the contained radioisotopes are extinguished (typically 10 half-lives) provides therapeutic advantages. If the resorption time is too short, the radionuclides/therapeutic agents may be released into surrounding tissue and cause off-target tissue damage. If the resorption rate is too long, healing in the region previously containing the tumor may be inhibited. Elements such as yttrium and phosphorus which have radioisotopes having a half-life greater than about two days and less than about 30 days are typically chosen as the constituent elements which emit therapeutic radiation.

The radiomedicine thermal gels discussed above are thus designed to emit high energy beta particles and/or alpha particles and/or gamma rays that have a relatively short penetration depth in tissue. While this is desired insofar as it optimizes tumor treatment while minimizing collateral tissue damage, it does pose a detection issue. Thus, it is advantageous to in certain instances introduce a second quantity of radioisotope characterized by emissions compatible with Single Photon Emission Computed Tomography (SPECT) or Positron Emission Tomography (PET) techniques that facilitate PET or SPECT imaging of the radiation distribution patterns in the patient and also aid in imaging of where the thermal gels deposit to ensure uniform deposition in and around the tumor site.

As with the treatment thermal gel, the composition of the imaging agent is selected such that the thermal gel emits a sufficient amount of positron emissions to facilitate PET imaging. In other words, the composition of the imaging agent is typically chosen so that its radiation may be tailored to deliver a radiation profile that is well suited for a particular imaging technique. For instance, when desired for use with PET imaging, the imaging thermal gel will typically include a short-lived positron emitter, such as ⁶⁴Cu (half-life of 12.7 hours) or ¹⁸F (half-life of 110 minutes) or the like. ⁶⁴Cu and ¹⁸F are particularly attractive positron emitters, as they have short half-lives and emit low energy positrons that annihilate with electrons to produce two 511 keV gammas, which facilitates PET imaging. If longer-lived positron emitters are desired, ⁸⁹Zr (half-life of 78.4 hours) or ¹²⁴I (half-life of 4.18 days) or the like may be selected.

In most instances, it is desirable to use a thermogel incorporating both treatment radioisotopes and imaging radioisotopes, such as a positron emitter (like ⁶⁴Cu) along with a beta and gamma emitter (like ⁸⁶Y) or a high-energy beta emitter (like ⁹⁰Y), such that the therapeutic treatment thermogel may themselves be directly imaged and tracked. In one such embodiment, a beta emitter and/or a low energy gamma emitting nuclide is incorporated along with a positron emitter into the thermogel

The treatment and imaging thermogel is typically introduced into the patient's body via catheter, injection or the like, gelation rapidly occurs in vivo, and the gel becomes lodged in the cancerous or tumor bearing tissue. It is understood that before the gelling of the thermogel is complete, the thermogel may fill voids in tissue left by surgery or enter interstitial portions of the treated tissue. The treatment and imaging thermogel is typically introduced as a liquid medium of sufficient density and viscosity such that the thermogel remains liquid during the administration procedure and gels rapidly upon introduction into the relatively warm target tissue.

The thermal gel has a composition that becomes more viscous when warmed to a higher temperature. Typically, thermal gel is more fluid at room temperatures and gels to become substantially more viscous, essentially behaving as a solid, at temperatures experienced in vivo. In one embodiment, a PLGA-g-PEG polymer was synthesized having a gelation onset temperature in phosphate buffered saline (PBS) of about 26° C. In one embodiment, the present novel technology relates to a method of preparing a radioactive insoluble particle suspension, in this example a radioactive yttrium particle suspension. Non-radioactive yttrium salts such as yttrium chloride, yttrium nitrate, yttrium sulfate, yttrium bromide and combinations thereof are combined with a soluble salt of radioactive yttrium-90, the latter typically being prepared by chemical separation of ⁹⁰Y from its parent isotope strontium-90 (⁹⁰Sr). Non-radioactive yttrium is combined with radioactive ⁹⁰Y to provide sufficient mass to yield discrete insoluble yttrium particles due to the vanishingly small masses of ⁹⁰Y required to provide therapeutic doses of radiation.

The yttrium salts are put into solution and subsequently combined with a solution of soluble phosphates salt(s), such as sodium phosphate, lithium phosphate, potassium phosphate, and combinations thereof, and having a stoichiometric excess of phosphate. The resultant admixture is maintained at a pH in the range of 1.5 to 8. The solutions are agitated, typically with continuous stirring and also rapidly heated in a closed vessel to about 150 degrees Celsius and held for from about one to about ten hours to yield a greater than about 99.99% conversion of soluble yttrium to insoluble YPO₄ as well as to achieve a desired particle size distribution, typically less than 2 microns in diameter, more typically in the range of 0.03 um to 10 um, still more typically in the range of 0.05 um to 3 um, and yet more typically in the range of 0.1 um to 2 um with a median particle size of about 0.2 um. Through careful control of mixing time, temperature, and concentration of the reactants, a specific, desired particle size distribution and/or particle shape distribution of YPO₄ particles suspended may be achieved. Likewise, once the YPO₄ particles are formed, the solution may be buffered with saline to achieve neutral pH suitable for direct injection into human or animal tissue.

Typically, the radioactive particle suspension has a mean particle size of less than 2 um. The radioactive particle suspension is typically characterized by at least 90 percent of the total particle volume having generally spherical particles in the range of 0.1 um to 2 um. Typically, the starting concentration of soluble yttrium in the combined solution is in the range of 0.05 to 1.0 mole/liter, more typically in the range of 0.05 to 0.3 mole/liter, and still more typically in the range of 0.08 to 0.3 mole/liter and the stoichiometric excess of phosphate ranges from 10% to 100%. More typically, the starting concentration of soluble yttrium in the combined solution is 0.08 moles/liter and the stoichiometric excess of phosphate is in the range of 5% to 100%, more typically about 10%, and still more typically about 25%.

In other embodiments, the radioactive metal cation is selected from members of the lanthanide series, such as Ce, Sm, Ho, Yb, Lu, and the like, and combinations thereof. In still other embodiments, the radioactive cation is selected from members of the metals and transition metals, such as Ga, In, Sn, Pb, Cu, Y, Sc, and the like and combinations thereof to yield insoluble or sparingly soluble transition metal phosphate(s). In yet other embodiments, the radioactive cation is selected from members of the alkali metals/alkali earth metals, such as Cs, Ra, Ca, Sr, Ba, and the like and combinations thereof, although these may be combined with insoluble inorganic compounds such as zeolites, as their phosphates may not be sufficiently insoluble. In still other embodiments, the radioactive cation is selected from members of the Actinide series, such as ²²⁵Ac, and in yet other embodiments, the radioactive cation is selected from members of the metals, transition metals, alkali metals, alkali earth metals, the Lanthanides, the Actinides, and combinations thereof. In some embodiments, the radioactive cation is selected from the members of the group including Y, La, Ce, Pr, Pm, Sm, Gd, Tb, Ho, Yb, Ce, Pb, Lu, Ac, Ca, Sr, Ba, Ra, Cs, Cu, Tc, Pd, Sn, Re, Au, and combinations thereof, while the phosphate functional group may include one or more radioisotopes of phosphorous, such as ³²P and ³³P (with ³¹P being the stable isotope). In some cases, the functional group may include an iodide wherein the iodide is a radioisotope, such as ¹²³I, ¹²⁴I, ¹²⁵I, and/or combinations thereof.

In operation, the particle suspension is formed by preparing the particle precursor solution of cation (for example, yttrium) salt and sodium phosphate to define an admixture. The admixture is then mixed and heated to yield a plurality of YPO₄ particles by controlled precipitation. The resulting YPO₄ particles are rinsed (typically multiple times, more typically three times) with a sterile phosphate buffered saline (PBS) solution and removing or adding PBS to achieve the final desired volume. The pH of the final solution is adjusted, such as by the addition of sodium hydroxide or the like, and then any excess solution is removed or sterile PBS is added to achieve a final desired volume. The YPO₄ particles are then suspended in a phosphate buffered saline solution at neutral pH, suitable for injection in vivo into human or animal tissue.

The yttrium phosphate particles are radioactive so as to serve as distributed sources of therapeutic radiation for treating cancerous tumors and other diseases, such as by adding a predetermined amount of soluble radioactive ⁹⁰Y isotope to the particle precursor solution, that becomes homogeneously incorporated into the insoluble yttrium phosphate particle matrix, solubility of less than about 10⁻⁶ mole/liter, more typically less than 10⁻²⁷ Ksp. The amount of radioactive yttrium (or like cation) is typically from about 100 μCi to 300 mCi; the specific amount needed varies for each patient application, Typically, the yttrium phosphate particle suspension has YPO₄ particle concentration in the range of 40 mg/ml to 125 mg/ml to facilitate imaging by x-ray computed tomography after being combined in a ratio of about 1 to 4 to 1 to 10 by volume with biocompatible hydrogel or other suitable liquid carrier solution for injection into human or animal tissue. Again, while the above example focuses on the yttrium cation, the technique may be adapted to accommodate others of the radioisotopes of interest.

It is well known in radiomedicine that certain types of cancerous tumors may be treated by the localized introduction of short-lived radioisotopes at the tumor site. One effective method of delivering such a radiation treatment is by introducing radioisotopes to the tumor site that emit gamma or beta radiation at therapeutic levels and intensities and that are suspended or otherwise contained in a thermal gel matrix, the gel being a liquid at room temperature that forms a solid gel upon warming to internal body temperature. The ⁹⁰Y or like radiotherapeutic radionuclide is typically introduced in the form of an insoluble stable oxide, phosphate, or the like, and suspended or dispersed in the thermal gel precursor, shortly before introduction into the patient's system. Typically, the predetermined radiotherapy treatment element has a short half-life, so that the radiation treatment is relatively short in duration; more typically, the predetermined element is selected such that it emits relatively high energy beta particles and/or alpha particles and/or gamma rays. For example, ⁹⁰Y has a half-life of 64 hours and emits beta particles with a mean energy of about 930 keV. This technique enjoys the advantage of using a thermally setting gel matrix prepared from stable, non-radioactive materials; the non-radioactive thermal gel may be safely stored for an indefinite period and combined with yttrium-90 or a like radiotherapy treatment element shortly before introduction into a patient's body.

Other potential radioisotopes that may be introduced via the thermal gel matrix include ¹³¹Cs, ¹²⁵I, ¹²¹Te, ¹⁰³Pd, ^{117m}Sn, and the like, which emit an Auger x-ray upon decay, and beta or beta/gamma emitters such as ³²P, ⁶⁷Cu, ¹²⁷Te, ¹³¹I, ¹⁷⁷Lu, ¹⁸⁶Re, ¹⁸⁸Re, and the like. In operation, ¹³¹Cs, ¹³¹I and/or ¹²⁵I and the like may be incorporated into the cage structure of a sodalite to yield an insoluble cesium sodalite compound, for example (such as via the hydrothermal synthesis of sodalite with Cs substituting for Na and/or I substituting for Cl in the crystal structure); likewise, ^{117m}Sn may be incorporated as an insoluble Sn apatite compound.

Still other potential radioisotopes that may be introduced via the thermal gel matrix include alpha-particle emitters such as ²¹²Pb, ²²³Ra, ²²⁵Ac, their respective decay products, and the like. These materials tend to form insoluble or sparingly soluble phosphates, and are thus good candidates for the novel treatments and materials described herein.

In an embodiment of the disclosure, a radionuclide selected for incorporation into a radio-thermogel may be chosen on the basis of the energy of the radiation emitted by the radionuclide. Lower energy emitters may be preferred for use when the radio-thermogel is placed in close proximity (about 2 mm, for example) to critical structures that may be damaged. In another embodiment, radionuclides that emit higher energy radiation may useful when placed farther from critical structures (greater than about 2 mm, for example). In another embodiment, a radio-thermogel may comprise both a beta-emitting radionuclide and a gamma-emitting radionuclide. In another embodiment, the radionuclide may be a beta- and gamma-emitter (for example, Lu-177, Sn-117m).

The thermogels disclosed herein can be combined with a radioactive component with or without one or more therapeutic agents. Such gels can provide a timed-release of the therapeutic agent into surrounding tissue. The timed release of the therapeutic agent may be a result of biodegradation of the gel or by diffusion of the therapeutic agent from the gel to the surrounding tissue.

It is believed that hydrolysis of the of the thermal gel may be an important process in the resorption of thermal gels. To develop thermal gels that are capable of gelling after introduction into the tissue being treating and to be resorbed during a therapeutically effective time frame several approaches are possible. Modification of the synthesis parameters of the thermal gelling polymer and/or modification of the ratios of the polymer backbone constituents (for example the ratios of lactide, glycolide, MPEG, and/or EPEG). It is also possible that mixing thermal gels with different polymeric backbones may provide thermal gel compositions that have gelling behaviors and resorption characteristics that are useful therapeutically. Batches of polymer were synthesized or combined in order to narrow the conditions necessary to obtain a gelation temperature below normal body temperature and a hydrolysis rate after gelation consistent with the desired resorption rate. The reaction conditions, polymer composition from Nuclear Magnetic Resonance (NMR) analysis, and gelation properties from dynamic rheology and hydrolysis rates at 37° C. are summarized below

### Methods

### Equipment

Electric Heating Mantle (Vevor, Model 98-I-AB-1000; S/N 231205 6090 27 0049)
Digital Heating Mantle (USA Labs, Model HMSC: 1000 ML; S/N 2023010084)
Freeze drier (HarvestRight)
Thermo Genesys 150 UV-Vis spectrophotometer
Nuclear Magnetic Resonance Spec (NMReady 60e, 60 MHz, Nanalysis)
Waters Breeze GPC system (model 1515 isocratic pump, model 2707 autosampler, model 2414
Refractive Index (RI) detector)
Rotary evaporator (Scilogex RE 100-Pro)
Analytical balance (Ohaus+/−0.1 mg)
Digital magnetic stirring reactor (Fisher Scientific* Isotemp* Digital Stirring Hotplate, 11-300-49SHP)
Refrigerated Circulator (Thermoscientific RTE-7, fill with 50:50 mixture of deionized water and commercial antifreeze)
Distillation head (Chemglass, 24/40 fittings)
Condenser (Chemglass, 24/40 fittings, ~20 cm long)
Vacuum distillation adapter (Chemglass, 24/40 with barb fitting for tube)
Inert gas/vacuum manifold (Chemglass, barbed)
Overhead Stirrer (Southwest Science)
Orbital titer plate shaker (IKA)
Reverse-osmosis water purifier (Cascada Model MK2)
Deionized water purifier (Easypure II system, Thermoscientific model #D7035)
Orbital agitating incubator (Southwest Science)

### Characterization Methods

### UV-Vis

A sample was scanned against a deionized water (DIH2O) blank by UV-Vis spectrophotometer (Thermofisher, Genesys 150) in the range of 190-900 nm. Spectra were collected in Absorbance mode.

### Gel-Permeation Chromatography External Standard (GPC-ES)

Samples were analyzed using GPC against polystyrene standards (external standard). The GPC system consisted of Waters 1515 Isocratic HPLC pump connected to Waters 2707 Autosampler and Waters 2414 Refractive Index Detector. Detector and columns held at 35° C. GPC analysis performed by injecting 100 μL of ~2.0 mg per mL polymer solution dissolved in 2.0 μm filtered tetrahydrofuran (THF). A run time of 60 minutes was set with the flow rate of 1 mL THF/min, and separation performed by a series of three GPC columns. The first column the samples passed through is a Phenomenex column Phenogel 5μ 50 A 300×7.8 mm, the second is Phenomenex column Phenogel 5μ 10E4A 300×7.8, and the last one is Aglient Resipore 300×7.5 mm 3 μm column. These samples were tested against Agilent Technologies EasiCal PS2 polystyrene standards. These standards were prepared according to manufacturer instructions using 0.2 μm filtered THF. Empower software was utilized to calculate number average (Mn) and weight average (Mw) molecular weight as well as polydispersity index.

### NMR

Samples (5-10 mg) were dissolved in 0.8 ml of deuterated chloroform (silver foil stabilized, Aldrich Cat #416754).

Processing was performed using ACDLabs Spectrus. Peaks were selected and integrated to identify peaks at 3.4 ppm (methyl —CH3, 3H), ~3.5-3.6 ppm (ethylene glycol CH2CH2O, 4H), ~4.7-4.9 ppm (glycolide CH2-C—O, 2H), and 5.1-5.3 ppm (lactide CHCH3-C═O, 1H), and terminal peak (CH3-C═O) at 4.2 ppm which corresponds to the last monomer in the chain though may be either lactide or glycolide. Notably additional polymer peak was observed at ~1.5-2.0 ppm (lactide CHCH3-C—O, 3H) though this was only used for establishing purity (% assigned peaks/total integration). For analysis, the peak integration at 3.4 ppm was fixed as equivalent to 3 hydrogens and each subsequent peak integration was normalized against this to obtain the relative molar ratios of lactide, glycolide, and ethylene glycol. The ratio of glycolide was set to “1” and the ratio of lactide and ethylene glycol was calculated respective to this ratio for composition ratio calculation.

### Gelation Tip-Test

0.5 mL thermogel solution was pipetted into an NMR tube. The tube was submerged into a 37° C. water bath until gelation occurred. Gelation time was established by pulling the tube from the bath every 10 seconds and conducting a tip test; it was said that gelation had occurred when the hydrogel did not fall or run when the tube was inverted.

### Rheology

Samples were tested for gelation by Rheology. Rheology was performed on AR2000 (TA instruments) with 60 mm 2 degree cone on a polymer dissolved in phosphate buffered saline (PBS) with stirring at 4° C. Viscosity of the solution at 0.1 (sec-1) and 5° C. was measured (1 minute peak hold 5 second test intervals). Rheology was performed by oscillating at constant 6.283 rad/s, 0.1% strain, in increments of 1° C. ranging from 5-45° C. with 1 minute of temperature equilibration at each point. The resultant G′/G″ was plotted against temperature. An increase in G′ and G″ with or without accompanying crossover of G′/G″ was taken to indicate onset of gelation with maximum achieved G′ and G″ (Pa) noted along with the respective temperature at which this maximum value occurred.

### Example 1. PLGA-G-PEG (LA:GA 50:50)

150 mL anhydrous toluene (TOL) was measured into a 150 mL volumetric flask using the following procedure: the flask was sealed with a rubber septum and vacuum purged using an 18G needle attached to a rubber hose. When negative pressure was achieved, the needle was removed. One end of a 24 inch double sided 20G cannula needle was inserted through the septa-seal all the way to the bottom of a bottle of anhydrous toluene (Acros cat #36441-0010). The other end of the cannula was inserted into the rubber septum of the volumetric flask. Argon was pumped into the bottle of toluene using the 18G needle attached to the rubber hose, causing the toluene to be transferred into the flask without coming into contact with air or moisture. The flask was filled to the 150 mL mark.

An acetone rinsed, 100° C. dried, desiccator cooled 4-neck 1000 mL RBF with magnetic stirbar was tared. 38.7 g EPEG (Aldrich cat #475696) was slowly pipetted in. The RBF was tared again. 4.5 g mPEG 750 (Acros cat #192325000) was added into the RBF. Added 150 ml toluene to the flask. An argon hose was connected to a stopcock attached to the side neck of the RBF and argon was gently pushed through the system (~40 cc/min) while a vacuum distillation apparatus was assembled. A distillation head was connected to the center neck. The condenser temperature on a coolant-recirculator was set to −10° C. A 500 mL 1-neck RBF along with a condenser drip-tip plus barbed side arm was attached to the end of the condenser head. The heating mantle's probe was pushed through a thermometer adapter and attached to a side neck in the RBF so that the tip of the probe was submerged in the PEG/TOL solution. The last neck was closed with a glass stopper. All attachments were secured with keck clips. The argon line was removed from the stopcock and attached to the barb on the drip-tip adapter; the stopcock was left open to vent argon. The argon was turned off and the stopcock was closed. Vacuum was applied through the hose attached to the drip-tip adapter. The PEG/TOL solution was set to stir ~200 RPM. The mantle heat was set to 50° C. In this manner, the PEGs were vacuum distilled until the toluene appeared to be gone (~1 hour). Afterward, the heat was turned off and the system was allowed to cool to room temperature under continuous vacuum. Once cooled, the system was backflushed with argon. Once pressure inside the system had equalized, the stopcock was opened to facilitate ventilation. Under continuous (~100 cc/min) argon flow, the distillation assembly was removed, keeping the stopcock in the side neck. Argon flow was slowed to ~25 cc/min. Through a funnel in the center neck, added 31.46 g D,L-Lactide (Ortec) and 25.31 g Glycolide (Ortec). A syringe was used to add in 6.45 mL 10% SnOct/TOL (w:v) solution. The argon was turned off. The reaction flask was vacuum purged for ~1 hour followed by argon backflush. Through a funnel in the center neck, added 150 mL anhydrous toluene (drawn from the bottle in the same manner as before). A 9″ glass Pasteur pipette was pushed through a thermometer adapter that was installed in the side-neck, replacing the stopcock, so that the tip of the pipette penetrated to ~1.5 inches below the surface of the reaction solution. A reflux condenser topped with a dry-trap was attached to the center neck. Coolant was circulated through the condenser at −10° C. A slow flow of argon was pumped through the pipette (~50 cc/min) with stirring for ~20 minutes. The Pasteur pipette/adapter were removed and replaced with the stopcock. Argon was pumped in through the stopcock at ~20 cc/min. The mantle temperature was set to 114° C. and stirring was set to a speed which produced a vortex with a visibly deep core and strong movement around the outer edges. Argon was allowed to continue flowing for another hour and then turned off. The reaction was allowed to continue for 24 hours. After 24 hours, the heat was turned off.

The reaction solution was evaporated under reduced pressure on a rotary evaporator until all toluene appeared to be gone. About 300 mL of acetone was added to the reaction flask and placed on room temperature orbital shaker until the material had dissolved. The solution was passed through a qualitative filter (Whatman) to remove any insoluble portions, then passed through a 1.2 μm nylon membrane filter, followed by a 0.8 μm nylon membrane filter, and finally a 0.45 μm nylon membrane filter using vacuum filtration. The filtered solution was poured directly into 2 L stirring hexane, the resulting solid was collected and then dried under vacuum at 55° C.

### Characterization

### NMR Analysis

| | Molar contribution (Methoxy = 1) | | | | | |
|---|---|---|---|---|---|---|
| Target | | | | Terminal | Relative ratio | |
| LA:GA | LA | GA | EG | (relative) | (LA:GA:EG) | Purity |
| 50:50 | 57 | 56 | 110 | 22 | 1.0:1:2.0 | 95% |

By GPC the Mn was determined to be 2089, the Mw was determined to be 4525, and the polydispersity index was determined to be 2.17. HNMR was performed and the ratio of components was determined as 1.0:1.0:2.0 (GA:LA:EG).

The polymer was dissolved in water at a concentration of 30% w/v and tested by thermal rheology. The viscosity at 5° C. was 0.04460 Pa·s. The Gelation Onset temperature was found to be 38.4° C. with a maximum G′ of 0.9888 Pa occurring at 44° C. and a maximum G″ of 2.083 Pa occurring at 43° C.

### Example 2. PLGA-G-PEG (LA:GA 65:35)

The same procedure as described in EXAMPLE 1 was followed with the following amounts of each monomer:
38.69 g EPEG, 4.51 g mPEG 750, 39.61 g D,L-Lactide, and 17.16 g Glycolide.

### Characterization

| | Molar contribution (Methoxy = 1) | | | | | |
|---|---|---|---|---|---|---|
| Target | | | | Terminal | Relative ratio | |
| LA:GA | LA | GA | EG | (relative) | (LA:GA:EG) | Purity |
| 65:35 | 74 | 42 | 116 | 29 | 1.8:1:2.7 | 95% |

By GPC the Mn was determined to be 2255 Da, the Mw was determined to be 4800 Da, and the polydispersity index was determined to be 2.03. HNMR was performed and the ratio of components was determined as 1.0:1.8:2.7 (GA:LA:EG).

The polymer was dissolved in water at a concentration of 30% w/v and tested by thermal rheology. The viscosity at 5° C. was 0.09056 Pa·s. The Gelation Onset temperature was found to be 30.2° C. with a maximum G′ of 19.21 Pa occurring at 34° C. and a maximum G″ of 38.50 Pa occurring at 34° C.

### Example 3. PLGA-G-PEG (LA:GA 75:25)

The same procedure as described in EXAMPLE 1 was followed with the following amounts of each monomer:
38.70 g EPEG, 4.51 g mPEG 750, 45.15 g D,L-Lactide, and 11.60 g Glycolide.

### Characterization

| | Molar contribution (Methoxy = 1) | | | | | |
|---|---|---|---|---|---|---|
| Target | | | | Terminal | Relative ratio | |
| LA:GA | LA | GA | EG | (relative) | (LA:GA:EG) | Purity |
| Target 75:25 | 84 | 29 | 107 | 20 | 2.9:1:3.7 | 94% |

By GPC the Mn was determined to be 1887 Da, the Mw was determined to be 3828 Da, and the polydispersity index was determined to be 2.03. HNMR was performed and the ratio of components was determined as 1.0:2.9:3.7 (GA:LA:EG).

The polymer was dissolved in water at a concentration of 30% w/v and tested by thermal rheology. The viscosity at 5° C. was 0.7123 Pa·s. The Onset temperature was found to be 33° C. with a maximum G′ of 6.556 Pa occurring at 36° C. and a maximum G″ of 17.66 Pa occurring at 36° C.

### Example 4. PLGA-G-PEG (LA:GA 85:15)

The same procedure as described in EXAMPLE 1 was followed with the following amounts of each monomer:
38.69 g EPEG, 4.51 g mPEG 750, 49.76 g D,L-Lactide, and 7.02 g Glycolide.

### Characterization

| | Molar contribution (Methoxy = 1) | | | | | |
|---|---|---|---|---|---|---|
| Target | | | | Terminal | Relative ratio | |
| LA:GA | LA | GA | EG | (relative) | (LA:GA:EG) | Purity |
| 85:15) | 98 | 19 | 114 | 19 | 4.0: 1:4.7 | 94% |

By GPC the Mn was determined to be 1972 Da, the Mw was determined to be 3831 Da, and the polydispersity index was determined to be 1.94. HNMR was performed and the ratio of components was determined as 1.0:4.0:4.7 (GA:LA:EG).

The polymer was dissolved in water at a concentration of 30% w/v and tested by thermal rheology. The viscosity at 5° C. was 0.04930 Pa·s. The Gelation Onset temperature was found to be 35.0° C. with a maximum G′ of 4.258 Pa occurring at 38° C. and a maximum G″ of 13.08 Pa occurring at 38° C.

### Example 5. PLA-G-PEG (LA:GA 100:0)

The same procedure as described in EXAMPLE 1 was followed with the following amounts of each monomer:
38.70 g EPEG, 4.50 g mPEG 750, and 59.10 g D,L-Lactide

### Characterization

| | Molar contribution (Methoxy = 1) | | | | | |
|---|---|---|---|---|---|---|
| Target | | | | Terminal | Relative ratio | |
| LA:GA | LA | GA | EG | (relative) | (LA:GA:EG) | Purity |
| 100:0 | 69 | NA | 70 | NA | NA | 94% |

By GPC the Mn was determined to be 2109 Da, the Mw was determined to be 4369 Da, and the polydispersity index was determined to be 2.07.

The polymer was dissolved in water at a concentration of 30% w/v and tested by thermal rheology. The viscosity at 5° C. was 0.04304 Pa·s. The Gelation Onset temperature was found to be 30.9° C. with a maximum G′ of 1.576 Pa occurring at 42° C. and a maximum G″ of 1.819 Pa occurring at 39° C.

### Example 6. Mixture Composition

A mixture of 75:25 LA:GA and 60:40 LA:GA was physically combined in aqueous solution and tested by rheology.

| | | | | | | | Gelation |
|---|---|---|---|---|---|---|---|
| | | | | Gelation | | | time |
| | Concentration | Viscosity | Viscosity | Onset | | | (from tip |
| Sample | in PBS | at 20° C. | at 5° C. | temperature | Max G′ | Max G″ | test) |
| Hydrogel | 25% (w:v) | 0.02236 | 0.03988 | 28.1° C. | 10.69 Pa | 20.14 Pa | 20 |
| blend: equal | | Pa · s | Pa · s | | (31° C.) | (32° C.) | seconds |
| parts of | 30% (w:v) | 0.03266 | 0.02353 | 28.7° C. | 12.55 Pa | 23.91 Pa | 15 |
| (75:25) and | | Pa · s | Pa · s | | (32° C.) | (32° C.) | seconds |
| (60:40) | 35% (w:v) | 0.03598 | 0.01790 | 29.2° C. | 14.92 Pa | 28.64 Pa | 10 |
| hydrogels | | Pa · s | Pa · s | | (32° C.) | (33° C.) | seconds |

### Resorption Study

### Method

Hydrogel solutions were prepared from the PLGA-g-PEG polymers at concentrations of 22%, 25%, and 30% (w:v).

Samples of each solution were prepared in triplicate as follows: The bottom tip of each dialysis cone (Pur-A-Lyzer Maxi 3500; Sigma-Aldrich cat no PURX35050; lot X032013) was cut off. An analytical balance was used to measure the mass of each cartridge. 2 mL of the indicated hydrogels were pipetted into the cartridges, and then the mass of each cartridge/hydrogel was measured. The mass of each empty cartridge was subtracted from the mass of the cartridge/hydrogel in order to determine the mass of the hydrogel. The mass of the hydrogel was multiplied by the actual concentration of the respective hydrogel in order to determine the beginning mass of polymer in each cartridge. The cartridges were dropped into 50 mL centrifuge tubes (Falcon) and then 40 mL of 37° C. DIH2O (measured using a graduated cylinder) was added to each tube. The tubes were placed into 37° C. incubator (no shaking) until gelled (approximately 30 minutes) and then rotation in the incubator was set to 80 RPM. At the designated time points, one tube at a time, the cartridges were removed from the tubes and then set aside while the tubes were re-capped and vortexed at high speed for ~5 seconds. 5 mL of the liquid from each tube was pipetted into a pre-tared ½ oz jar (U-Line) and capped loosely; the rest of the liquid was decanted into waste. The cartridges were placed back into the Falcon tubes and 40 mL of fresh 37° C. DIH2O was added. The tubes were replaced in the incubator with 80 RPM shaking until the next measurement. The jars were lyophilized and then measured on the same analytical balance that was used to measure their tare-mass. The difference between the tare mass and the mass of the lyophilized jars was multiplied by 8 in order to calculate the mass of polymer that had eluted (resorbed). The average mass eluted was divided by the average beginning mass for each sample in order to calculate the percent eluted (resorbed). Following the same method, repeated measurements were made at the specified time intervals for a total of 8 weeks. At the end of the 8 weeks, the dialysis cartridges were lyophilized and then measured on an analytical balance in order to obtain the final mass of polymer within each cartridge.

### Results

The resultant biodegradation/resorption rate was determined gravimetrically

Data from Weekly Media Draws: PLGA-g-PEG (L:G 65:35)

| | L:G 65:35 22% | | L:G 65:35 25% | | L:G 65:35 30% | |
|---|---|---|---|---|---|---|
| | Initial mass | | | | | |
| | 0.4480 g | | 0.4992 g | | 0.6051 g | |
| | Average | | Average | | Average | |
| | mass | Cumulative | mass | Cumulative | mass | Cumulative |
| | eluted | Average % | eluted | Average % | eluted | Average % |
| | (g) | eluted | (g) | eluted | (g) | eluted |
| 1 day | 0.0672 ± 0.006 | 15.00% | 0.0533 ± 0.041 | 10.68% | 0.0835 ± 0.015 | 13.79% |
| 3 days | 0.0205 ± 0.019 | 19.58% | 0.0371 ± 0.005 | 18.11% | 0.0341 ± 0.024 | 19.43% |
| 7 days | 0.0320 ± 0.024 | 26.73% | 0.0285 ± 0.006 | 23.82% | 0.0235 ± 0.005 | 23.31% |
| 10 days | 0.0075 ± 0.004 | 28.39% | 0.0016 ± 0.001 | 24.15% | 0.0123 ± 0.005 | 25.34% |
| 14 days | 0.0080 ± 0.006 | 30.18% | 0.0037 ± 0.006 | 24.89% | 0.0117 ± 0.006 | 27.28% |
| 21 days | 0.0117 ± 0.005 | 32.80% | 0.0075 ± 0.002 | 26.39% | 0.0149 ± 0.007 | 29.75% |
| 28 days | 0.0229 ± 0.004 | 37.92% | 0.0389 ± 0.003 | 34.19% | 0.0437 ± 0.002 | 36.97% |
| 35 days | 0.0603 ± 0.004 | 51.37% | 0.0555 ± 0.010 | 45.30% | 0.0757 ± 0.004 | 49.49% |
| 42 days | 0.0523 ± 0.001 | 63.04% | 0.0555 ± 0.005 | 56.41% | 0.0603 ± 0.005 | 59.45% |
| 49 days | 0.0235 ± 0.016 | 68.27% | 0.0499 ± 0.004 | 66.40% | 0.0645 ± 0.006 | 70.12% |
| 56 days | 0.0261 ± 0.002 | 74.11% | 0.0320 ± 0.004 | 72.81% | 0.0373 ± 0.002 | 76.28% |

Data from Weekly Media Draws: PLGA-g-PEG (L:G 75:25)

| | L:G 75:25 22% | | L:G 75:25 25% | | L:G 75:25 30% | |
|---|---|---|---|---|---|---|
| | Initial mass | | | | | |
| | 0.4438 g | | 0.5000 g | | 0.5997 g | |
| | Average | | Average | | Average | |
| | mass | Cumulative | mass | Cumulative | mass | Cumulative |
| | eluted | Average % | eluted | Average % | eluted | Average % |
| | (g) | eluted | (g) | eluted | (g) | eluted |
| 1 day | 0.0501 ± 0.004 | 11.30% | 0.0325 ± 0.017 | 6.51% | 0.0349 ± 0.027 | 5.83% |
| 3 days | 0.0195 ± 0.008 | 15.68% | 0.0128 ± 0.007 | 9.07% | 0.0101 ± 0.009 | 7.51% |
| 7 days | 0.0192 ± 0.002 | 20.01% | 0.0123 ± 0.007 | 11.52% | 0.0141 ± 0.006 | 9.87% |
| 10 days | 0.0043 ± 0.006 | 20.97% | 0.0083 ± 0.004 | 13.17% | 0.0043 ± 0.004 | 10.58% |
| 14 days | 0.0035 ± 0.007 | 21.75% | 0.0072 ± 0.002 | 14.61% | 0.0069 ± 0.007 | 11.74% |
| 21 days | 0.0069 ± 0.005 | 23.31% | 0.0179 ± 0.004 | 18.19% | 0.0653 ± 0.031 | 22.63% |
| 28 days | 0.0099 ± 0.003 | 25.54% | 0.0408 ± 0.016 | 26.35% | 0.0552 ± 0.017 | 31.84% |
| 35 days | 0.0376 ± 0.007 | 34.01% | 0.0507 ± 0.006 | 36.48% | 0.0701 ± 0.006 | 43.53% |
| 42 days | 0.0685 ± 0.003 | 49.45% | 0.0864 ± 0.001 | 53.76% | 0.0939 ± 0.009 | 59.19% |
| 49 days | 0.0456 ± 0.003 | 59.73% | 0.0560 ± 0.000 | 64.96% | 0.0579 ± 0.007 | 68.83% |
| 56 days | 0.0467 ± 0.004 | 70.24% | 0.0581 + 0.001 | 76.59% | 0.0547 ± 0.005 | 79.95% |

Data from Weekly Media Draws: PLGA-g-PEG (L:G 85:15)

| | L:G 85:15 22% | | L:G 85:15 25% | | L:G 85:15 30% | |
|---|---|---|---|---|---|---|
| | Initial mass | | | | | |
| | 0.4385 g | | 0.4846 g | | 0.5981 g | |
| | Average | | Average | | Average | |
| | mass | Cumulative | mass | Cumulative | mass | Cumulative |
| | eluted | Average % | eluted | Average % | eluted | Average % |
| | (g) | eluted | (g) | eluted | (g) | eluted |
| 1 day | 0.0800 ± 0.011 | 18.24% | 0.0661 ± 0.004 | 13.65% | 0.0600 ± 0.013 | 10.03% |
| 3 days | 0.0221 ± 0.009 | 23.29% | 0.0099 ± 0.008 | 15.68% | 0.0099 ± 0.007 | 11.68% |
| 7 days | 0.0144 ± 0.001 | 26.58% | 0.0096 ± 0.011 | 17.66% | 0.0157 ± 0.005 | 14.31% |
| 10 days | 0.0067 ± 0.002 | 28.10% | 0.0075 ± 0.003 | 19.20% | 0.0053 ± 0.005 | 15.20% |
| 14 days | 0.0027 ± 0.004 | 28.70% | 0.0051 ± 0.005 | 20.25% | 0.0077 ± 0.005 | 16.50% |
| 21 days | 0.0013 ± 0.001 | 29.01% | 0.0019 ± 0.002 | 20.64% | 0.0056 ± 0.003 | 17.43% |
| 28 days | 0.0013 ± 0.002 | 29.31% | 0.0024 ± 0.002 | 21.13% | 0.0088 ± 0.013 | 18.90% |
| 35 days | 0.0019 ± 0.000 | 29.74% | 0.0075 ± 0.009 | 22.67% | 0.0280 ± 0.027 | 23.59% |
| 42 days | 0.0003 ± 0.000 | 29.80% | 0.0352 ± 0.051 | 29.94% | 0.0776 ± 0.036 | 36.56% |
| 49 days | 0.0021 ± 0.002 | 30.29% | 0.0372 ± 0.032 | 37.61% | 0.0712 ± 0.034 | 48.46% |
| 56 days | 0.0080 ± 0.002 | 32.11% | 0.0371 ± 0.011 | 45.26% | 0.0712 ± 0.009 | 60.37% |

Total mass lost, as calculated by measuring the mass of polymer remaining in the cartridge at the end of the 8-week study.

| Sample/ | | initial mass of | final mass of | | |
|---|---|---|---|---|---|
| concentration | | polymer | polymer | Total mass lost | Total percent lost |
| 65% | 22% | 0.4480 g ± 0.0026 g | 0.0304 g ± 0.0013 g | 0.416 g ± 0.0019 g | 93.21% ± 0.25% |
| lactide | 25% | 0.4992 g ± 0.0049 g | 0.0312 g ± 0.0066 g | 0.4680 g ± 0.0044 g | 93.75% ± 1.27% |
| | 30% | 0.6051 g ± 0.0082 g | 0.0454 g ± 0.0034 g | 0.5597 g ± 0.0061 g | 92.51% ± 0.49% |
| 75% | 22% | 0.4438 g ± 0.0033 g | 0.0713 g ± 0.0064 g | 0.3725 g ± 0.0032 g | 83.95% ± 1.32% |
| lactide | 25% | 0.5000 g ± 0.0075 g | 0.0500 g ± 0.0439 g | 0.4499 g ± 0.0462 g | 89.97% ± 8.77% |
| | 30% | 0.5997 g ± 0.0018 g | 0.0754 g ± 0.0095 g | 0.5242 g ± 0.0109 g | 87.42% ± 1.61% |
| 85% | 22% | 0.4385 g ± 0.0039 g | 0.2569 g ± 0.0051 g | 0.1816 g ± 0.0070 g | 41.40% ± 1.37% |
| lactide | 25% | 0.4846 g ± 0.0305 g | 0.2875 g ± 0.0160 g | 0.1970 g ± 0.0428 g | 40.41% ± 6.53% |
| | 30% | 0.5981 g ± 0.0033 g | 0.3226 g ± 0.0163 g | 0.2755 g ± 0.0166 g | 46.07% ± 2.74% |

### Resorption Test NMR

Example NMR properties of eluted materials for PLGA-g-PEG (LA:GA 75:25) as calculated from NMR

| | | Molar contribution (Methoxy = 1) | | | | Relative |
|---|---|---|---|---|---|---|
| | | | | | Terminal | ratio |
| Sample | | LA | GA | EG | (relative) | (LA:GA:EG) |
| 2 | hours | 3 | 1 | 52 | 8 | 5.1:1:93.7 |
| 1 | day | 9 | 2 | 124 | 16 | 4.5:1:64.8 |
| 2 | days | 17 | 5 | 116 | 18 | 3.5:1:23.8 |
| 3-7 | days | 29 | 8 | 83 | 23 | 3.8:1:10.7 |
| 8 | days | 48 | 13 | 83 | 36 | 3.6:1:6.4 |
| 9 | days | 22 | 6 | 31 | 12 | 3.4:1:4.9 |
| 10 | days | 34 | 11 | 48 | 19 | 3.1:1:4.4 |
| 11 | days | 24 | 7 | 32 | 13 | 3.3:1:4.3 |
| 12-14 | days | 38 | 11 | 49 | 23 | 3.5:1:4.5 |
| 15 | days | 12 | 4 | 17 | 8 | 3.4:1:4.6 |
| 16 | days | 44 | 12 | 61 | 30 | 3.6:1:5.0 |
| 17 | days | 20 | 6 | 28 | 16 | 3.2:1:4.5 |
| 18 | days | 15 | 4 | 18 | 11 | 3.4:1:4.0 |
| 19-21 | days | 44 | 13 | 42 | 26 | 3.5:1:3.4 |
| 22 | days | 22 | 6 | 30 | 14 | 3.4:1:4.6 |
| 23 | days | 30 | 8 | 30 | 17 | 3.6:1:3.6 |
| 24 | days | 21 | 7 | 19 | 12 | 3.1:1:2.8 |
| 25 | days | 21 | 7 | 19 | 12 | 3.1:1:2.8 |
| 26-28 | days | 25 | 7 | 43 | 17 | 3.4:1:5.8 |
| 29 | days | 27 | 8 | 38 | 16 | 3.2:1:4.5 |
| 30 | days | 29 | 9 | 33 | 16 | 3.2:1:3.7 |
| 31-37 | days | 51 | 14 | 49 | 37 | 3.6:1:3.4 |
| 38-43 | days | 38 | 11 | 74 | 38 | 3.4:1:6.8 |
| 44-51 | days | 45 | 14 | 78 | 113 | 3.3:1:5.6 |
| 52-58 | days | 123 | 32 | 83 | 263 | 3.8:1:2.6 |

The NMR results, although varied in composition, were consistent with the resultant released materials being lactic acid, glycolic acid, poly(ethylene glycol) and various compositions of oligomers of said compounds. This may indicate biocompatibility of the resorption by-products as all these components are well tolerated by the human body.

Several non-limiting embodiments of the disclosure are described in the following clauses.
1. A biodegradable thermogel suspension that forms a gel at a temperature of about 20° C. to about 40° C. comprising:
one or more PLGA-g-PEG polymers, which are synthesized under conditions substantially free of water molecules and oxygen molecules, then dissolved in an aqueous buffer solution;
wherein the molar ratio of lactate monomer (LA) to glycolate monomer (GA) in the one or more PLGA-g-PEG polymers is individually from about 65:35 to about 85:15; and
wherein the biodegradable thermogel biodegrades with a half-life of from about 1 day to about 365 days or about 10 to about 50 days.
2. The biodegradable thermogel of clause 1, wherein the biodegradable thermogel comprises a single PLGA-g-PEG polymer.
3. The biodegradable thermogel of clause 1 or 2, wherein the molar ratio of LA:GA is about 65:35.
4. The biodegradable thermogel of clause 1 or 2, wherein the molar ratio of LA:GA is about 75:25.
5. The biodegradable thermogel of clause 1 or 2, wherein the molar ratio of LA:GA is about 85:15.
6. The biodegradable thermogel of any one of the preceding biodegradable thermogel clauses, wherein the ratio of EPEG+mPEG to PLGA in the one or more PLGA-g-PEG polymers is individually from 1:99 to 99:1 or about 1:1 to about 1:2.
7. The biodegradable thermogel of any one of the preceding biodegradable thermogel clauses wherein, the ratio of mPEG to EPEG in the one or more PLGA-g-PEG polymers is individually from 1:99 to 99:1, or about 1:8 to about 1:10
8. The biodegradable thermogel suspension of any one of the preceding biodegradable thermogel clauses, wherein the one or more PLGA-g-PEG polymers have a weight average molecule weight (Mw) of about 100 Da to about 10,000 Da or about 500 Da to about 8000 Da, or about 1000 Da to about 7000, or about 1500 Da to about 5000 Da.
9. The biodegradable thermogel suspension of any one of the preceding biodegradable thermogel clauses, wherein the one or more PLGA-g-PEG polymers independently have a number average molecule weight (Mn) of about 100 Da to about 10,000 Da or about 500 Da to about 8000 Da, or about 1000 Da to about 7000, or about 1500 Da to about 5000 Da.
10. The biodegradable thermogel suspension of any one of the preceding biodegradable thermogel clauses, wherein the weight of the one or more PLGA-g-PEG polymers is in the range of about 15 wt % to about 40 wt % or about 20 wt % to about 35 wt % in aqueous buffer solution.
11. The biodegradable thermogel suspension of any one of the preceding biodegradable thermogel clauses, wherein the thermogel suspension has a pH between 1.5 and 8.0
12. A biodegradable radioactive thermogel suspension that forms a gel at a temperature of about 20° C. to about 40° C. comprising:
one or more PLGA-g-PEG polymers, which are synthesized under conditions substantially free of water molecules and oxygen molecules, then dissolved in an aqueous buffer solution;
wherein the molar ratio of lactate monomer to glycolate monomer in the one or more PLGA-g-PEG polymers is individually from about 65:35 to about 85:15; and
a plurality of chemically isolated radioactive particles suspended in the thermogel, wherein each chemically isolated radioactive particle includes a cation and a functional group;
wherein the plurality of chemically isolated radioactive particles are between 0.03 μm and 10 μm in diameter;
wherein the chemically isolated radioactive particles have water solubility of less than 1×10⁻⁶ mole/liter; and
wherein the biodegradable radioactive thermogel biodegrades with a half-life of from about 1 day to about 365 days or about 10 to about 50 days.
13. The biodegradable radioactive thermogel suspension of clause 12, wherein the biodegradable radioactive thermogel comprises a single PLGA-g-PEG polymer.
14. The biodegradable radioactive thermogel of clause 1 or 2, wherein the molar ratio of LA:GA is about 65:35.
15. The biodegradable radioactive thermogel of clause 1 or 2, wherein the molar ratio of LA:GA is about 75:25.
16. The biodegradable radioactive thermogel of clause 1 or 2, wherein the molar ratio of LA:GA is about 85:15.
17. The biodegradable radioactive thermogel of any one of the preceding biodegradable radioactive thermogel clauses wherein the ratio of EPEG+mPEG to PLGA in the one or more PLGA-g-PEG polymers is individually from 1:99 to 99:1 or from about 1:1 to about 1:2.
18. The biodegradable radioactive thermogel of any one of the preceding biodegradable radioactive thermogel clauses, wherein, the ratio of mPEG to EPEG in the one or more PLGA-g-PEG polymers is individually from 1:99 to 99:1.
19. The biodegradable radioactive thermogel suspension of any one of the preceding clauses wherein the one or more polymers have a weight average molecule weight (Mw) of about 100 Da to about 10,000 Da or about 500 Da to about 8000 Da, or about 1000 Da to about 7000, or about 1500 Da to about 5000 Da.
20. The biodegradable radioactive thermogel suspension of any one of the preceding biodegradable radioactive thermogel clauses, wherein the one or more polymers have a number average molecule weight (Mn) of about 100 Da to about 10,000 Da or about 500 Da to about 8000 Da, or about 1000 Da to about 7000, or about 1500 Da to about 5000 Da.
21. The biodegradable radioactive thermogel suspension of any one of the preceding biodegradable radioactive thermogel clauses, wherein the radioactive particles are combined with the thermogel to yield a final concentration of thermogelling polymer in the range of about 15 wt % to 40 wt % in aqueous buffer solution.
22. The biodegradable radioactive thermogel suspension of any one of the preceding biodegradable radioactive thermogel clauses, wherein the biodegradable radioactive thermogel suspension has a pH between 1.5 and 8.0.
23. The biodegradable radioactive thermogel suspension of any one of the preceding biodegradable radioactive thermogel clauses wherein the cation is selected from the group consisting of Y, La, Ce, Pr, Pm, Sm, Gd, Tb, Ho, Yb, Cs, Pb, Lu, Ac, Ca, Sr, Ba, Ra, Cu, Tc, Pd, Sn, Re, Au, and combinations thereof; and wherein the functional group includes phosphorous isotopes selected from the group consisting of ³¹P, ³²P, ³³P, and combinations thereof.
24. The biodegradable radioactive thermogel suspension of any one of the preceding biodegradable radioactive thermogel clauses, wherein the cation is selected from the group consisting of Y, Ho, Yb, Ce, Pb, Pd, Cs, Ac, Sm, Lu, Sc, Ca, Sr, Ba, and combinations thereof.
25. The biodegradable radioactive thermogel suspension of any one of the preceding biodegradable radioactive thermogel clauses, wherein the functional group includes isotopes selected from the group consisting of ³¹p, ³²P, ³³P, ¹²³I, ¹²⁴I, ¹²⁵I, and combinations thereof.
26. The biodegradable radioactive thermogel suspension of any one of the preceding biodegradable radioactive thermogel clauses, wherein the cation is ⁹⁰Y and the functional group is a phosphate, wherein the phosphorus of the phosphate includes one or more isotopes selected from the group consisting of ³¹P, ³²P, and ³³P.
27. The biodegradable radioactive thermogel suspension of clause 26, wherein the phosphorus of the phosphate is ³¹P at its natural abundance.
28. The biodegradable radioactive thermogel suspension of any one of the preceding biodegradable radioactive thermogel clauses, wherein the chemically isolated radioactive particle concentration is in the range of 3 mg/ml to 100 mg/ml.
29. The biodegradable radioactive thermogel suspension of any one of the preceding biodegradable radioactive thermogel clauses, wherein the chemically isolated radioactive particles provide a dosage of between 30 μCi and 300 mCi.
30. The biodegradable radioactive thermogel suspension of any one of the preceding biodegradable radioactive thermogel clauses wherein the functional group is selected from the group consisting of apatite, sodalite, iodide, hydride, and combinations thereof.
31. The biodegradable radioactive thermogel of any one of the preceding biodegradable radioactive thermogel clauses, wherein the radioactive particles emit radiation with an energy of about 0.01 MeV to about 6 MeV
32. The biodegradable radioactive thermogel of any one of the preceding biodegradable radioactive thermogel clauses, wherein the radioactive particles emit beta radiation and gamma radiation.
33. The biodegradable radioactive thermogel of any one of the preceding biodegradable radioactive thermogel clauses, wherein the gelled biodegradable radioactive thermogel biodegrades with a half-life of biodegradation of about the same as the half-life of the radioactive particles.
34. The biodegradable radioactive thermogel of any one of the preceding biodegradable radioactive thermogel clauses, wherein the gelled biodegradable radioactive thermogel biodegrades with a half-life of about 5 days to about 90 days.
35. The biodegradable radioactive thermogel of any one of the preceding biodegradable radioactive thermogel clauses, further comprising a therapeutic agent selected from the group consisting of antibodies, small interfering RNAs (siRNAs), imaging agents, metallic nanoparticles, anti-cancer agents, radiosensitizers, hormones, antibiotics, analgesics, anti-inflammatory agents, immunoactivators, growth factors, gene therapy agents, oligonucleotides, antisense nucleotides, peptides, and proteins, enzyme inhibitors, chelators, nanocarriers, probiotics, photodynamic therapy agents, microRNA (miRNA), cytokines, neurotransmitter modulators, antioxidants, biomimetic peptides, exosomes, sensory modulation agents, aptamers, medicinal compounds, and combinations thereof
36. A therapeutic depot formed when the biodegradable radioactive thermogel of any one of the preceding biodegradable radioactive thermogel clauses forms a gel after administration to a warm-blooded patient.

What is claimed:

1. A biodegradable thermogel suspension that forms a gel at a temperature of about 20° C. to about 40° C. comprising:
one or more PLGA-g-PEG polymers, which are synthesized under conditions substantially free of water molecules and oxygen molecules, then dissolved in an aqueous buffer solution;
wherein the molar ratio of lactate monomer (LA) to glycolate monomer (GA) in the one or more PLGA-g-PEG polymers is individually from about 65:35 to about 85:15; and
wherein the biodegradable thermogel biodegrades with a half-life of from about 1 day to about 365 days or about 10 to about 50 days.

2. The biodegradable thermogel of claim 1, wherein the biodegradable thermogel comprises a single PLGA-g-PEG polymer.

3. The biodegradable thermogel of claim 1, wherein the ratio of EPEG+mPEG to PLGA in the one or more PLGA-g-PEG polymers is individually from 1:99 to 99:1.

4. The biodegradable thermogel of claim 3, wherein the ratio of EPEG+mPEG to PLGA in the one or more PLGA-g-PEG polymers is individually from about 1:1 to about 1:2.

5. The biodegradable thermogel of claim 1 wherein, the ratio of mPEG to EPEG in the one or more PLGA-g-PEG polymers is individually from 1:99 to 99:1.

6. The biodegradable thermogel of claim 5 wherein, the ratio of mPEG to EPEG in the one or more PLGA-g-PEG polymers is individually from about 1:8 to about 1:10.

7. The biodegradable thermogel suspension of claim 1, wherein the one or more PLGA-g-PEG polymers have a weight average molecule weight (Mw) of about 100 Da to about 10,000 Da or about 500 Da to about 8000 Da, or about 1000 Da to about 7000 Da, or about 1500 Da to about 5000 Da.

8. The biodegradable thermogel suspension of claim 1, wherein the one or more PLGA-g-PEG polymers independently have a number average molecule weight (Mn) of about 100 Da to about 10,000 Da or about 500 Da to about 8000 Da, or about 700 Da to about 7000 Da, or about 1000 Da to about 5000 Da.

9. The biodegradable thermogel suspension of claim 1, wherein the weight of the one or more PLGA-g-PEG polymers is in the range of about 15 wt % to about 40 wt % or about 20 wt % to about 35 wt % in aqueous buffer solution.

10. The biodegradable thermogel suspension of claim 1, wherein the thermogel suspension has a pH between 1.5 and 8.0.

11. A biodegradable radioactive thermogel suspension that forms a gel at a temperature of about 20° C. to about 40° C. comprising:
one or more PLGA-g-PEG polymers, which are synthesized under conditions substantially free of water molecules and oxygen molecules, dissolved in an aqueous buffer solution;
wherein the molar ratio of lactate monomer to glycolate monomer in the one or more PLGA-g-PEG polymers is individually from about 65:35 to about 85:15; and
a plurality of chemically isolated radioactive particles suspended in the thermogel, wherein each chemically isolated radioactive particle includes a cation and a functional group;
wherein the plurality of chemically isolated radioactive particles are between 0.03 μm and 10 μm in diameter;
wherein the chemically isolated radioactive particles have water solubility of less than 1×10⁻⁶ mole/liter; and
wherein the biodegradable radioactive thermogel biodegrades with a half-life of from about 1 day to about 365 days, or about 10 days to about 50 days.

12. The biodegradable radioactive thermogel suspension of claim 11, wherein the biodegradable radioactive thermogel comprises a single PLGA-g-PEG polymer.

13. The biodegradable radioactive thermogel of claim 11, wherein the ratio of EPEG+mPEG to PLGA in the one or more PLGA-g-PEG polymers is individually from 1:99 to 99:1.

14. The biodegradable radioactive thermogel of claim 13, wherein the ratio of EPEG+mPEG to PLGA in the one or more PLGA-g-PEG polymers is individually from about 1:1 to about 1:2.

15. The biodegradable radioactive thermogel of claim 11, wherein, the ratio of mPEG to EPEG in the one or more PLGA-g-PEG polymers is from 1:99 to 99:1.

16. The biodegradable radioactive thermogel of claim 15, wherein, the ratio of mPEG to EPEG in the one or more PLGA-g-PEG polymers is individually from about 1:8 to about 1:10.

17. The biodegradable radioactive thermogel suspension of claim 11, wherein the one or more polymers have a weight average molecule weight (Mw) of about 100 Da to about 10,000 Da or about 500 Da to about 8000 Da, or about 1000 Da to about 7000, or about 1500 Da to about 5000 Da.

18. The biodegradable radioactive thermogel suspension of claim 11, wherein the one or more polymers have a number average molecule weight (Mn) of about 100 Da to about 10,000 Da or about 500 Da to about 8000 Da, or about 700 Da to about 7000 Da, or about 1000 Da to about 5000 Da.

19. The biodegradable radioactive thermogel suspension of claim 11, wherein the radioactive particles are combined with the thermogel to yield a final concentration of thermogelling polymer in the range of about 15 wt % to 40 wt % in aqueous buffer solution.

20. The biodegradable radioactive thermogel suspension of claim 11, wherein the biodegradable radioactive thermogel suspension has a pH between 1.5 and 8.0.

21. The biodegradable radioactive thermogel suspension of claim 1 wherein the cation is selected from the group consisting of Y, La, Ce, Pr, Pm, Sm, Gd, Tb, Ho, Yb, Cs, Pb, Lu, Ac, Ca, Sr, Ba, Ra, Cu, Tc, Pd, Sn, Re, Au, and combinations thereof, and wherein the functional group includes phosphorous isotopes selected from the group consisting of ³¹P, ³²P, ³³P, and combinations thereof.

22. The biodegradable radioactive thermogel suspension of claim 21, wherein the cation is selected from the group consisting of Y, Ho, Yb, Ce, Pb, Pd, Cs, Ac, Sm, Lu, Sc, Ca, Sr, Ba, and combinations thereof.

23. The biodegradable radioactive thermogel suspension of claim 11, wherein the functional group includes isotopes selected from the group consisting of ³¹P, ³²P, ³³P, ¹²³I, ¹²⁴I, ¹²⁵I, and combinations thereof.

24. The biodegradable radioactive thermogel suspension of claim 11, wherein the cation is ⁹⁰Y and the functional group is a phosphate, wherein the phosphorus of the phosphate includes one or more isotopes selected from the group consisting of ³¹P, ³²P, and ³³P.

25. The biodegradable radioactive thermogel suspension of claim 20, wherein the phosphorus of the phosphate is ³¹P at its natural abundance.

26. The biodegradable radioactive thermogel suspension of claim 11, wherein the chemically isolated radioactive particle concentration is in the range of 3 mg/ml to 100 mg/ml.

27. The biodegradable radioactive thermogel suspension of claim 11, wherein the chemically isolated radioactive particles provide a dosage of between 30 μCi and 300 mCi.

28. The biodegradable radioactive thermogel suspension of claim 11 wherein the functional group is selected from the group consisting of apatite, sodalite, iodide, hydride, and combinations thereof.

29. The biodegradable radioactive thermogel of claim 11, wherein the radioactive particles emit radiation with an energy of about 0.01 MeV to about 6 MeV

30. The biodegradable radioactive thermogel of claim 11, wherein the radioactive particles emit beta radiation and gamma radiation.

31. The biodegradable radioactive thermogel of claim 11, wherein the gelled biodegradable radioactive thermogel biodegrades with a half-life of biodegradation of about the same as the half-live of the radioactive particles.

32. The biodegradable radioactive thermogel of any of claim 11, wherein the gelled biodegradable radioactive thermogel biodegrades with a half-life of about 5 days to about 90 days.

33. The biodegradable radioactive thermogel of claim 11, further comprising a therapeutic agent selected from the group consisting of antibodies, small interfering RNAs (siRNAs), imaging agents, metallic nanoparticles, anti-cancer agents, radiosensitizers, hormones, antibiotics, analgesics, anti-inflammatory agents, immunoactivators, growth factors, gene therapy agents, oligonucleotides, antisense nucleotides, peptides, and proteins, enzyme inhibitors, chelators, nanocarriers, probiotics, photodynamic therapy agents, microRNAs (miRNAs), cytokines, neurotransmitter modulators, antioxidants, biomimetic peptides, exosomes, sensory modulation agents, aptamers, medicinal compounds, and combinations thereof

34. A therapeutic depot formed when the biodegradable radioactive thermogel of claim 11 forms a gel after introduction into a patient.
